(19) United States
(12) Patent Application Publication
(10) Pub. No.: US 2017/0007173 A1
(43) Pub. Date: Jan. 12, 2017

(54) SYSTEM FOR POLYPHASIC SLEEP MANAGEMENT, METHOD OF ITS OPERATION, DEVICE FOR SLEEP ANALYSIS, METHOD OF CURRENT SLEEP PHASE CLASSIFICATION AND USE OF THE SYSTEM AND THE DEVICE IN POLYPHASIC SLEEP MANAGEMENT

(71) Applicant: INTELICLINIC SPOLKA Z OGRANICZONA ODPOWIEDZIALNOSCIA, Warszawa (PL)

(72) Inventors: Kamil ADAMCZYK, Lublin (PL); Janusz FRACZEK, Warszawa (PL); Krzysztof CHOJNOWSKI, Wloclawek (PL)

(21) Appl. No.: 15/114,256

(22) PCT Filed: Jan. 26, 2015
(86) PCT No.: PCT/IB2015/050570

§ 371 Date: Jul. 26, 2016

(30) Foreign Application Priority Data
Jan. 27, 2014 (PL) PL406957

(51) Int. Cl. A61B 5/00; A61M 21/02; A61B 5/0496; A61B 5/11; A61B 5/0478; A61B 5/0492

(52) U.S. Cl. CPC A61B 5/4812; A61B 5/002; A61B 5/0478; A61B 5/0492; A61B 5/0496; A61B 5/1118; A61B 5/4815; A61B 5/486; A61B 5/6803; A61B 5/725; A61M 21/02; A61B 5/7264; A61B 2562/0219; A61M 2021/0022

(57) ABSTRACT

The object of the present invention is a system for polyphasic sleep management, characterized by the fact it comprises components such as electrodes for measuring a biological signal; a biological amplifier for amplifying the biological signal; a microcontroller for controlling individual system components, such as a biological amplifier and an accelerometer, as well as for communication with an external device; an accelerometer for collecting data relating to user's movement frequency; an external device, such as a computer, tablet and a mobile phone and a method of its operation. The invention also relates to a device for sleep analysis, a method of current sleep phase classification and use of the system and the device for polyphasic sleep management.

The object of the invention is a system for polyphasic sleep management, a method of its operation, a device for sleep analysis, a method of current sleep phase classification and use of the system and the device in polyphasic sleep management. The present invention is used in order to increase polyphasic sleep efficiency, to adapt the polyphasic sleep mode to the individual characteristics of the user's body, to establish a schedule for the polyphasic sleep, to inform the user of the forthcoming activities related to the polyphasic sleep. The invention may also be used to avoid the unpleasant sensations associated with passing from the state of sleep to the state of wakefulness.

Sleep is a functional state of the central nervous system, periodically occurring and passing in a circadian rhythm, during which abolition of consciousness (with the exception of lucid dream) and lack of motion occur. Sleep is characterized by receding under the influence of external factors.

Monophasic sleep is the standard sleep mode. This is the most common sleeping method. It involves a continuous sleep (over 4 hours or shorter) once per day, usually during night time. The recommended duration for sleep of this type is 7-9 hours, depending on the organism.

Polyphasic sleep is a practice of iterative, intermittent sleep during 24 hours. The name does not refer to a specific sleep model, but describes a set of different sleep plans, the common feature of which being the division of monophasic sleep into shorter periods of sleep/napping following one another at intervals.

Monophasic sleep differs from polyphasic sleep primarily in the periods (frequency and length), during which we remain in the state of sleep. As a consequence, the individual phases of sleep in these two different modes are characterized by different content as well.

According to the stages of sleep classification of the American Academy of Sleep Medicine (AASM), monophasic sleep is divided into the following stages of sleep:

1. W phase (wakefulness), phase of wakefulness—blinking in EOG, will-dependent, rapid eye movements, slow, floating eye movements during somnolence; high voltage score in EMG, occurrence of will-dependent body movements; EEG with eyes open: low voltage mixed activity with dominance of beta waves (>13 Hz); with eyes closed: low voltage mixed activity with dominance of alpha waves (8-13 Hz) representing more than 50% of the scoring time.

2. Sleep with slow eye movements (abbreviation: NREM—non-rapid eye movement); other names: deep sleep, slow-wave sleep. In this phase, Δ waves in the brain's electrical activity appear. Based on the fraction of slow waves it was divided into 2 stages:
stage 1, wherein awareness of environmental stimuli gradually decreases, slow, floating eye movements in EOG, medium voltage score in EMG, EEG score with low voltage mixed activity with dominance of theta waves (4-8 Hz) representing more than 50% of the scoring time.
stage 2 is characterized by lack of reactivity to stimuli, decline in and subsequently lack of eye movements in EOG, low voltage score in EMG, EEG score with low voltage mixed activity with dominance of theta waves, presence of EEG graph elements characteristic for the N2 stage: sleep spindles and K complexes.
stage 3—lack of eye movements in EOG, EOG score contains high voltage slow waves from EEG, low voltage score in EMG, high voltage slow delta waves (0-2 Hz) in EEG, representing more than 20% of the scoring time.

3. R phase—sleep with rapid eye movements (REM—rapid eye movement); other names: shallow sleep, paradoxical sleep. In this phase, dreams most often occur, there is also a complete muscle relaxation, which is why a human dreaming of movement does not move.

Rapid eye movements in EOG, the lowest amplitude of EMG scoring in EMG, episodic (phasic) muscle contractions, low voltage mixed activity in EEG with dominance of theta and beta waves, presence of EEG graph elements characteristic for the R stage: sawtooth waves.

Therefore, to characterize the architecture, that is the structure of sleep, overnight analysis of three physiological parameters is required:

1. bioelectric brain function, which is evaluated with electroencephalography (EEG), with electrodes placed on the skin of the head, over the left and right brain hemispheres, in the frontal, central and occipital areas,

2. eye movements, which are analyzed with electrooculography (EOG)—electrodes are placed laterally and below the left eye, as well as laterally and above the right eye,

3. muscle tone, which is analyzed with electromyography (EMG), with electrodes placed above the chin muscles.

Sleep begins with the NREM phase, properly with a duration of 80-100 minutes, followed by the REM sleep phase, lasting about 15 minutes. In adults, a cycle of this type is repeated 4-5 times.

Along with the duration of sleep:
the fraction of the deepest stadium of slow-wave sleep (with the highest activity of Δ waves) decreases;
the duration of the REM phase increases, usually lasting around 40 minutes by the end of the night.

Polyphasic Sleep

REM is considered to be the most effective phase of sleep, wherein the brain regenerates the most. A human in monophasic sleep, periodically enters and comes out of REM sleep, passing through NREM sleep phases. Sleep never begins with the REM phase and in order to achieve it, one needs to go through all of NREM phases first.

Polyphasic sleep presupposes a decrease in the amount of sleep, exactly at the expense of the NREM phase (in different ways, depending on the mode of polyphasic sleep), so that the body sleeps utilizing mainly REM sleep and so that in the moment of falling asleep it enters the REM phase directly, omitting the NREM phase.

Beneficial Effects of Transition from Monophasic Sleep to Polyphasic One

1) time saving. The recommended daily (24 hours) duration of monophasic sleep is about 7-9 hours. With polyphasic sleeping it is possible to shorten sleep time to 6.5 to 2 hours per 24 hours, thus gaining several hours of activity/wakefulness per day.

2) According to studies, frequent changes in typical sleeping hours, that is a situation taking place during shift working, is detrimental to health. Due to the transition to polyphasic sleep, there is an opportunity to regulate sleep, even in case of work with frequent shifting of day and nighttime. This is much healthier for the body.

Due to sleeping problems resulting from civilizational, environmental changes, the necessity of prolonging the time of activity or of adapting to the changing sleeping hours, there is a need in the art for methods of transition to polyphasic sleep and management thereof, as well as systems and devices enabling the implementation of these methods.

And so, in an American patent publication no. U.S. Pat. No. 8,398,538, a method of sleep management and a system and a device used in the method were disclosed, wherein patient's movements, lighting, temperature and sounds in the vicinity of the patient are analyzed, with the use of sensors either built into the bed or situated in the vicinity of the patient's bed. The system is directed mainly to children and elderly and its major use is assisting the parents/caretakers of the children in optimizing sleep behavior of children, who have completely different sleep behavior than adults.

Patent applications US 20130002435 and WO 2012170586 refer to a device, named Jawbone Up, having a form of a band worn on a wrist for activity monitoring during day and night. During the day, the band using an inside located accelerometer, monitors the wearer movements, calculates the number of steps taken, the amount of calories burned, etc. Whereas during the night, the band monitors sleep, using a method called ‘actigraphy’, which assumes that during REM sleep the body is paralyzed and the body does not move. In this way, the band distinguishes between REM sleep phase and NREM and based on the data collected by the accelerometer wakes the user in the most suitable moment, before falling into deep sleep.

Smartphone and tablet applications, such as Sleep Cycle (iOS) or Sleep as Android (Android) are also known, for monitoring of users sleep. Those applications monitor sleep using a phone/tablet built-in accelerometer; or the one in the Jawbone, FitBit wristband, using the abovementioned phenomenon, the so-called ‘actigraphy’, that is a characterization of sleep (wakefulness/sleep) and sleep phases (REM/NREM) using solely body movements of the studied person. Those applications, based on the above study, record sleep history and wake in the most suitable moment therefor (before entering deep sleep).

The above described methods, systems and devices do not utilize monitoring of parameters, such as EEG (measuring of brain waves), EOG (measuring of eye movements) or EMG (measuring of muscle tone). They do not include a polyphasic sleep mode either, wherein the user is gradually guided from monophasic sleep to polyphasic sleep, nor do they establish sleeping schedules suited to the user's body on the basis of user data. The present invention is directed mainly to adults, from 18 to 65 years and its main assumption is to facilitate the transition from monophasic sleep to polyphasic sleep for the user.

The device for sleep measurement according to the present invention utilizes electrodes placed on the user's forehead. With these, an electrical signal for brain activity (EEG—electroencephalographic study), eye movements (EOG—electrooculography) and muscle tone (EMG—electromyography) is collected from user's body. In the device according to the invention there is also an accelerometer, collecting data of patient's movements from the head. The device, apart from measuring sleep phases (wakefulness/sleep and REM/NREM phase) and the function of waking the user in the most suitable moment (before falling into deep sleep) has, as the first device in the world, a polyphasic sleep mode. In this mode the user has a sleep and napping plan established, customized to the sleep model he chooses. The used application automatically adapts the sleep mode, based on data collected from previous sleeps, to the current needs of the user's body, monitoring the regularity of sleep on a constant basis. The application stepwise, gradually changes the user's sleep mode from monophasic to polyphasic, so that the transition period is as unburdensome as possible for him, because of too little sleep.

The device according to the present invention, as the first device on the market, employs the technology of EEG, EOG and EMG measurements and the accelerometer for collection of data, which will subsequently be processed using artificial intelligence methods in order to create a unique polyphasic sleep mode, customized for an individual user.

Sleep analysis based solely on user's body movements is much less precise than the one based on user's body movements, brain wave measurement, eye movements and muscle tone. In the device according to the invention all four of the above parameters are measured, in contrast to the known devices, measuring only user's body movements.

Furthermore, the known devices, in contrast to the device according to the invention, do not facilitate waking using light. The present device in the form of a head mask contains built-in LEDs, which lighten up 5 minutes before the planned waking, thus adapting the user's eyes to light, wherein often the light itself is enough to very gently wake the user from sleeping, without the need to use an alarm.

The starting point for general use electronic devices, whose function involves monitoring, optimization of sleep, are the polysomnographs commonly used in modern medicine. Those devices collect biological signals from the patient's head, using techniques such as: EEG (electroencephalography)—brainwaves measurement, EOG (electrooculography)—measurement of frequency of eye movements and EMG (electromyography)—measurement of muscle contractions. Based on collected data the physician manually records the above waveforms and on their basis concludes about sleep-associated disease/irregularity diagnosis.

The signals collected by a polysomnograph also allow an experienced physician to determine in which sleep phase the described irregularities occur.

Apart from the above described devices, whose manufacturers claim that they are capable of characterizing human sleep with data collected with an accelerometer, there have also recently appeared devices attempting to epitomize the function of a polysomnograph. And thus, a device named ‘Multi-modal sleep system’ disclosed in an American patent application no. US 20130060097 A1 under the principle of a polysomnograph operation, collects an EEG signal from the patient's forehead with 3 electrodes (2 signal electrodes and ground). The EEG signal together with the polysomnograph signal serves in the device to automatically determine which sleep phase the user is in. However, none of the described devices provide measurements as precise as the device according to the present invention, which uses all 4 abovementioned parameters and enables the user to pass from the monophasic sleep mode to polyphasic sleep.

Currently, individuals desiring to change their wakefulness rhythm to polyphasic sleep are forced into manual sleep management. This involves adapting proper lengths of the main nighttime sleep and the lengths of naps during the day.

Therefore, the object of the present invention was to develop a method of sleep management, a system and a device used in the method, enabling the creation of a unique polyphasic sleep mode customized to an individual user, and an easy, gradual transition from monophasic sleep mode to polyphasic, as well as monitoring sleep regularity on a constant basis.

The object is realized by a system for polyphasic sleep management, comprising components, such as electrodes for measuring a biological signal; a biological amplifier for amplifying the biological signal; a microcontroller for controlling individual system components, such as a biological amplifier and an accelerometer, as well as for communication with an external device; an accelerometer for collecting data relating to user's movement frequency; an external device, such as a computer, tablet and a mobile phone.

Preferably, the system according to the present invention comprises at least 3 electrodes, two frontal ones for measuring a biological signal, connected to the input of the biological amplifier and a central one connected to the ground of the amplifier.

Preferably, the system according to the present invention additionally comprises two zygomatic electrodes and two temporal electrodes.

Preferably, the system according to the present invention is characterized by the fact that in the biological amplifier the signal is initially high-pass filtered by the input followers and amplified; afterwards it is band-stop filtered with an active filter of the ‘double-T’ type, followed by low-pass filtering with a quaternary filter of Butterworth characteristic, which simultaneously additionally amplifies the signal, and the last amplifying stage is realized using a bilateral switch.

Preferably, in the system according to the present invention, the microcontroller classifies the current sleep phase based on the biological signal received by the analog-to-digital converter and the data from the accelerometer, and sends the obtained results to the external device.

Preferably, in the system according to the present invention, the microcontroller communicates with the external device via Bluetooth Low Energy (BLE) interface.

Preferably, in the system according to the present invention, the accelerometer additionally wakes the microcontroller from sleep mode.

The object of the present invention is also a device for sleep analysis comprising a mask, acting as a carrier component, for mounting on the user's head; at least one PCB plate, at least three electrodes, two frontal ones, for measuring a biological signal connected to the input of a biological amplifier and a central one connected to the ground of the amplifier, and a power source, such as an accumulator battery; wherein the PCB plate contains components such as a microcontroller for controlling individual system components, such as a biological amplifier and an accelerometer, as well as for communication with an external device; a biological amplifier for amplifying the biological signal; an accelerometer for collecting data relating to user's movement frequency.

Preferably, the device according to the present invention additionally comprises an integrated antenna and/or at least one light source (8), such as a one-color LED, a multicolored RGB LED, a lightbulb; and/or a vibrating component; and/or two zygomatic electrodes and two temporal electrodes.

Preferably, in the system according to the present invention, components such as the electrodes for measuring a biological signal; the biological amplifier for amplifying the biological signal; the microcontroller for controlling the individual system components such as the biological amplifier and the accelerometer, as well as for communication with an external device; the accelerometer for collecting data relating to user's movement frequency; are integrated in one device for sleep analysis.

The invention also relates to a method of operation of the abovementioned system comprising steps, wherein the system is activated, the device for sleep analysis is initialized; connection is established between the device for sleep analysis and an external device, which subsequently sends configuration data such as the waking hour, duration of the waking buffer, the waking method; the device for sleep analysis is mounted on the user's head; electrodes contact with the user's skin is tested; a biological signal is measured; the current sleep phase is classified; waking is commenced in the suitable sleep phase; data is sent to the external device; the device for sleep analysis is brought into sleep mode, wherein it awaits re-initialization.

Preferably, in the method according to the present invention, the device for sleep analysis is initialized by an event from the accelerometer.

Preferably, in the method according to the present invention, the connection between the device for sleep analysis and the external device is established using a wireless Bluetooth technology.

Preferably, in the method according to the present invention, the waking is commenced after the first REM phase detected during the course of the waking buffer or after reaching operation limit for the device.

The invention also relates to a method of current sleep phase classification comprising steps, wherein electrodes test is performed in order to confirm the possibility of measuring a biological signal; amplification by the biological amplifier is regulated based on a test run, so that the maximal signal value does not exceed the acceptable voltage value for the analog-to-digital converter; an n-seconds signal fragment is acquired; an amplitude spectrum for the acquired signal is calculated k-times using Fast Fourier Transform, FFT; the calculated spectra are averaged and normalized; a feature vector for the averaged and normalized spectra and the accelerometer data is generated; the obtained feature vector is compared with standards for individual sleep phases; the sleep phase corresponding to the standard with the highest correlation with the feature vector is selected.

Preferably, in case of negative result of the electrodes test, a feature vector is generated only from accelerometer data, followed by comparison of the obtained feature vector with the standards for individual sleep phases and the sleep phase corresponding to the standard with the highest correlation with the feature vector is selected.

The object of the present invention is also a use of the abovementioned system for polyphasic sleep management.

The object of the present invention is also a use of the abovementioned device for polyphasic sleep management.

The object of the invention is illustrated with embodiments on figures, where:

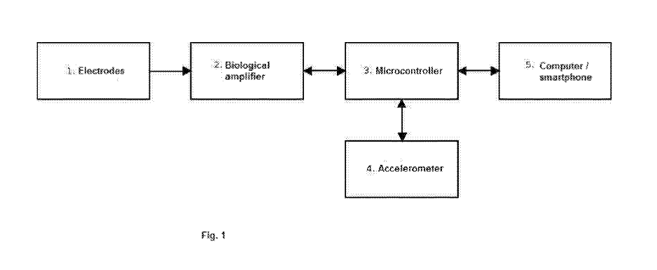

FIG. 1 shows the structure of the system for polyphasic sleep management according to the present invention,

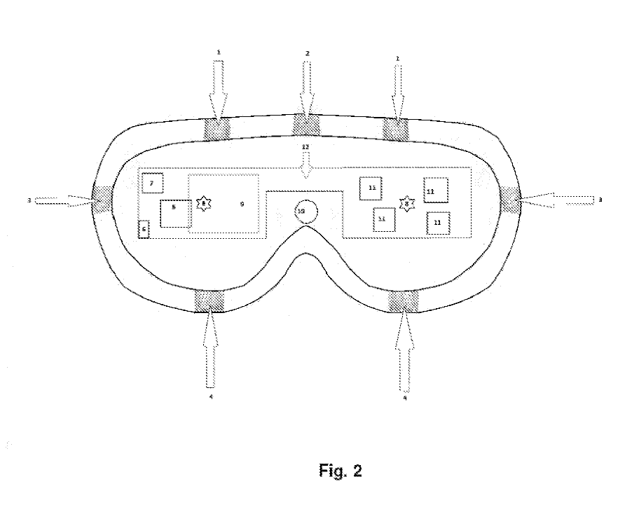

FIG. 2 shows the device for sleep analysis according to the present invention,

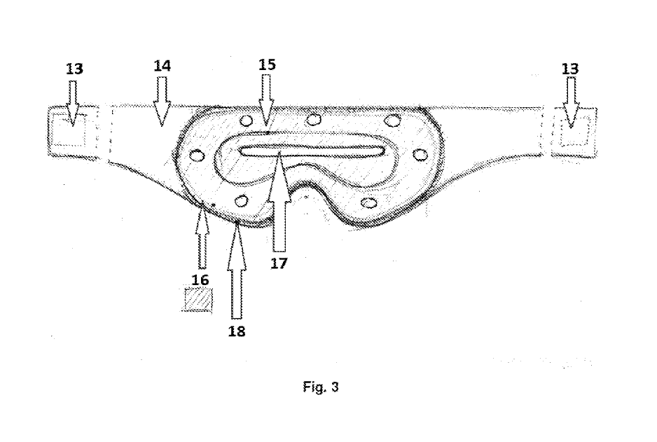

FIG. 3 shows an embodiment of electrodes in the device for sleep analysis according to the present invention;

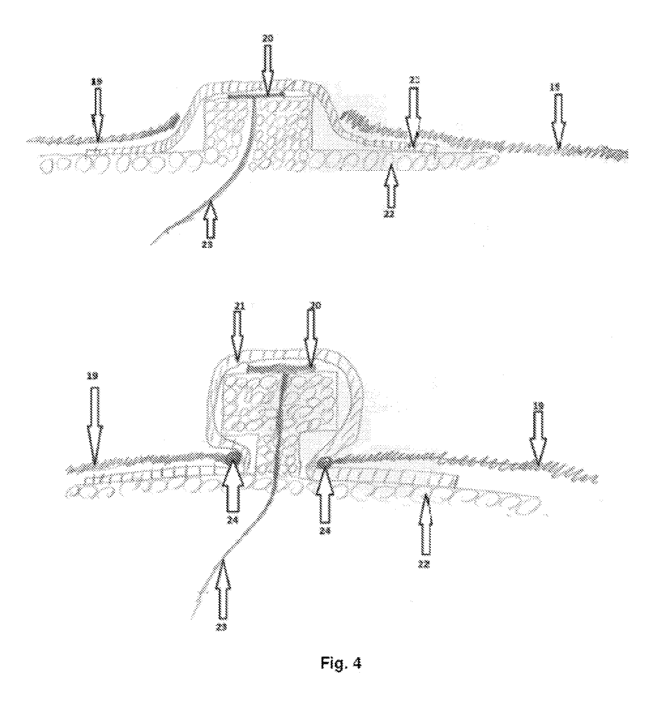

FIG. 4 shows a different embodiment of electrodes in the device for sleep analysis according to the present invention;

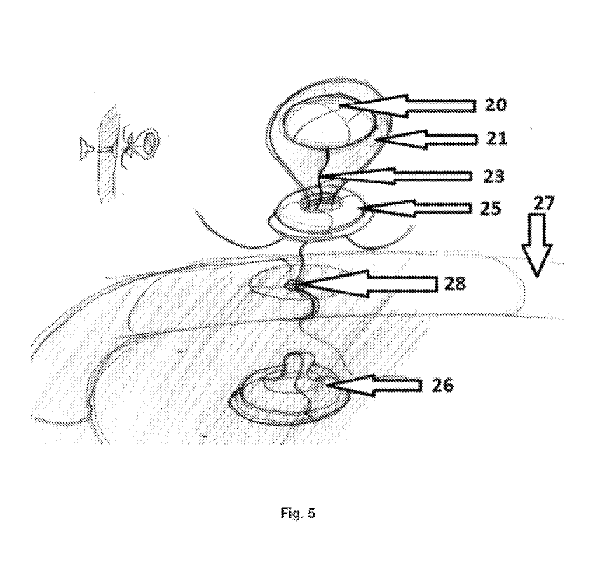

FIG. 5 shows the method of operation of the system for polyphasic sleep management according to the present invention,

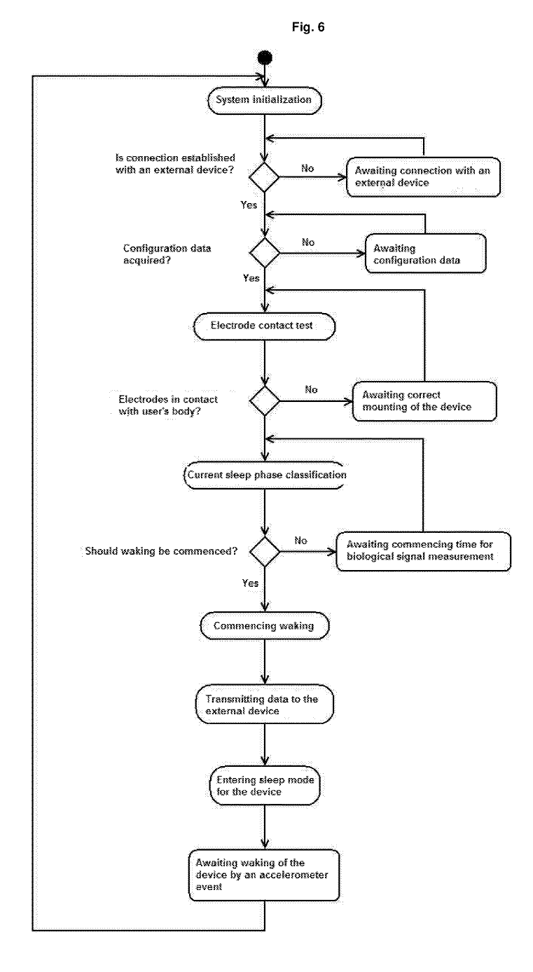

FIG. 6 shows the method of current sleep phase classification according to the present invention.

FIG. 1 shows the structure of the system for polyphasic sleep management according to the present invention. The components forming the system, constituting the complete, analog-to-digital processing path for biological signals, are as follows:

1. Electrodes

The basic three electrodes are placed on the user's forehead. The central electrode is connected to the ground of the amplifier and the distal electrodes reach the differential input thereof. The voltage between them is measured. The system may also be equipped with additional electrodes, placed under the eyes (on cheekbones) and at the outer corners of the eyes (temporal areas).

2. Biological Amplifier

In one embodiment of the system according to the invention, the biological amplifier is constructed based on an instrumental amplifier. The signal is initially high-pass filtered by the input followers and amplified. Afterwards it is band-stop filtered with an active filter of the ‘double-T’ type, finally followed by low-pass filtering with a quaternary filter of Butterworth characteristic, which simultaneously additionally amplifies the signal. The last amplifying stage is adjusted within five amplifications. This has been realized using a bilateral switch, which includes in the loop of the operational amplifier resistors of different denominations. After amplification, the signal is provided to the input of the microcontroller's ADC converter.

3. Microcontroller

The microcontroller has two important functions in the system. The first is controlling individual components of the system (biological amplifier, accelerometer) and communication with the outside world (external device), e.g. via a Bluetooth Low Energy (BLE) interface. The second task of the microcontroller is predicting the current sleep phase based on the biological signal received with the built-in analog-to-digital converter and accelerometer data (more in the operation algorithm chapter).

4. Accelerometer

The accelerometer is used for collecting data on the studied person's movement frequency. The data is then taking part in the prediction of the current sleep phase realized by the microcontroller. The accelerometer is additionally used for waking the microcontroller from sleep mode in order to prolong working time on a single battery.

5. Computer/Smartphone

The results of calculations realized by the device are sent to an external device, which may be a personal computer, a smartphone or another device capable of establishing a connection, e.g. equipped with a BLE interface. Transmission of the results is used for visualization and archiving thereof.

In a preferable embodiment, the electrodes for measuring a biological signal, the biological amplifier for amplifying the biological signal, the microcontroller for controlling individual system components, such as the biological amplifier and the accelerometer, as well as for communication with an external device; the accelerometer for collecting data relating to user's movement frequency, are integrated in one device for sleep analysis, preferably having a form of a face mask.

Diagrams for the device for sleep analysis according to the present invention are illustrated on FIG. 2 and FIG. 3. The device for sleep analysis comprises a mask, acting as a carrier component, for mounting on the user's head, at least one PCB plate 12, preferably flexible, at least three electrodes, two frontal ones 1 for measuring a biological signal, connected to the input of a biological amplifier 11 and a central one 2 connected to the ground of the amplifier 11, and a power source 9, such as an accumulator battery. The PCB plate 12 contains components such as a microcontroller 5 for controlling individual system components, such as a biological amplifier 11 and an accelerometer 7, as well as for communication with an external device; a biological amplifier 11 for amplifying the biological signal, and an accelerometer 7 for collecting data relating to user's movement frequency. The device may additionally comprise an integrated antenna 6, at least one LED 8, preferably two LEDs 8, and additionally a vibrating component 10. In a preferable embodiment the device additionally comprises two zygomatic 4 electrodes and two temporal 3 electrodes.

The invention is used by putting the mask on the face during sleeping, such that the electrodes 1, 2, 3, 4 adhere to the locations on the forehead, temples or viscerocranium. During sleep the mask is sending signals to a near-located computer/mobile phone/tablet. When it is time to wake the user from sleep, the mask wakes the user using light generated by the light source 8 (e.g. such as a LED of a single color, a multicolored RGB LED, a lightbulb) placed in the mask, and vibration generated by the vibration motor 10 placed in the mask, and an application on the phone/computer/tablet serves as an alarm clock and emits a sound signal.

The object of the invention is a method of human polyphasic sleep management and an electronic sleeping mask together with an application for a computer/tablet/mobile phone for human polyphasic sleep management. The invention also relates to a system comprising the sleeping mask, a computer/tablet/mobile phone, wherein the suitable application for using the mask will be installed, and a suitable PC computer together with an application allowing for reading the statistics from the computer/tablet/mobile phone. The system according to the present invention may comprise all three abovementioned components together (the mask, the computer/tablet/mobile phone, the PC computer), as well as one or two therefrom having all its functionalities and any combination of these components.

The mask according to the present invention, shown on FIG. 2 and FIG. 3 comprises four components: a viscoelastic foam filling 15, a PCB (Printed Circuit Board) plate 12 together with electrodes 1, 2, 3, 4 and wires connecting the plate with the electrodes, a thin plastic plate and a textile covering together with a wide elastic band 14 mounting the mask on the face and hook-and-loop fasteners 13 and fabric joining individual components 16 and 18.

Due to the use of viscoelastic foam, shape of the mask adapts to the curvature of the user's face. The outer curvature of the viscoelastic foam resembles ski goggles, since it has a spherical plane. The inner surface is a viscerocranium contour from the middle of the forehead at the top to the cheekbones at the bottom with a cutout for the nose. The inside of the mask is concave, adjusted in shape to the PCB plate form with a groove 17 near the outside surface for mounting the plastic plate. The PCB plate is separated from the user's face by a stiffening plate made of plastic and polycarbonate. The textile covering enters into the groove near the outside surface, encasing the outside part of the mask but not encasing the plastic plate.

On the inside of the covering (from the face side) there is preferably a total of seven electrodes 1, 2, 3, 4 mounted, made of a conductive fabric 21, in connection with conductive threads. The central frontal electrode 2 is the ground for the user, and the two frontal distal electrodes 1 serve to amplify the bioelectric signal (EEG, EOG, EMG) in the first channel of the biological amplifier. Two temporal electrodes 3 are used for acquiring the bioelectric signal in the second channel, and two electrodes placed under the eyes 4 (on zygomatic bones) register bioelectric signals in the third channel.

In one embodiment illustrated on FIG. 4 the electrodes are made in form of a surface on the covering 19, sewn in e.g. with a conducting thread from the face side. The electrodes pass through to the other side of the covering, where they contact the contact fields 20, e.g. in the form of a gilded plate, permanently located in the mask. Wires 23 depart from these contact fields 20, carrying the biological signal to the input of the biological amplifier 11. In another embodiment, the electrode may be additionally fixed using a clamping ring 24. In a further embodiment illustrated on FIG. 5, the electrode may be fixed to the covering using latches 25 and 26.

The mask is mounted on the face using a wide elastic band fastened on the side, at the temple area. The mask must adhere to the face, so that the electrodes are in tight contact with the face.

If the mask was deactivated with a hardware switch, it should be activated using the same, to provide power from an accumulator battery or a battery to the system's electronics. If the mask was deactivated using the application (software), it is enough to move it for it to turn itself on automatically—e.g. by lifting it off the table. This is realized by the built-in accelerometer, activating (waking) the processor having registered acceleration above a predefined threshold.

Then, the phone/tablet/computer should have its auto-lock deactivated and should preferably be connected to a charger for the night. One should open the suitable application, log in, select one of the main menu options, set the time till waking or activate the automatic mode. When the device pairs (establishes a connection with the phone) then the LEDs located therein will light up in blue. The suitable option for commencing sleep tracking should be loaded. The LEDs will light up e.g. in red. When red LEDs are light up the device should be put on the head, so that the electrodes touch the forehead and the other areas where the electrodes are placed. If the device detects a correct skin contact it will indicate as such with a flash of green LEDs. The phone should be relatively close, so that the device do not lose the link with the phone.

Impedance control for the electrodes is activated by providing to the patient's ground (amplifier) a signal from a test generator. Then, the ground electrode still has an average potential of zero, but with a superimposed fast-changing generator signal with a frequency of 500 Hz. The test signal, through the patient's body, reaches the electrodes, and thus the input of the biological amplifier. Directly on the inputs of the biological amplifier selective amplifiers are placed, receiving only the test signal, which from their inputs is provided to the input of the ADC converter of the microcontroller. The better the contact for the electrode, the higher the amplitude for the signal received therewith. A weak signal, with the same amplitude for both electrodes for the given channel indicates with high probability poor contact for the ground electrode. With negligible probability this may indicate contact deterioration for two input electrodes of the amplifier, in equal measure.

Signal processing is realized in several steps. In the first step amplitude spectra are calculated with the FFT method from signal time courses of a few seconds. When a particular number of spectra are collected, they are averaged. In the next step, feature vectors are calculated for the classification algorithm, which is an artificial neural network, a support vector machine (SVM) or a decision tree. The feature vector is based on signal amplitude for bands corresponding to particular frequency ranges corresponding to brainwaves (alpha, beta, gamma, delta)—the EEG signal, eye movements—the EOG signal and muscle movement—the EMG signal. To the thus generated feature vector information is added relating to the frequency of user's movement during sleep—received from the accelerometer. Thus prepared feature vector is processed by the classification algorithm, whereby a sleep phase in which the studied person is at the moment is determined.

The device is powered from an accumulator battery or a battery.

The new method of polyphasic sleep management involves an automatic adjustment of the duration of the main nighttime sleep, the duration and number of individual naps and time intervals between sleep/napping episodes.

The automatic method is based on EEG, EOG and EMG signals and accelerometer signal measurement. Based on the above data the signal is processed in order to determine which sleep phase the user is in. The system aims at switching the user from monophasic sleep, composed of REM, NREM 1, 2 and 3 sleep phases to polyphasic sleep, wherein sleep is composed mainly of REM sleep. The system automatically sets a schedule for the transition from monophasic sleep to polyphasic sleep and monitors the efficacy of polyphasic sleep (the REM to NREM sleep ratio).

Methods of Operation of the System and Sleep Phase Classification

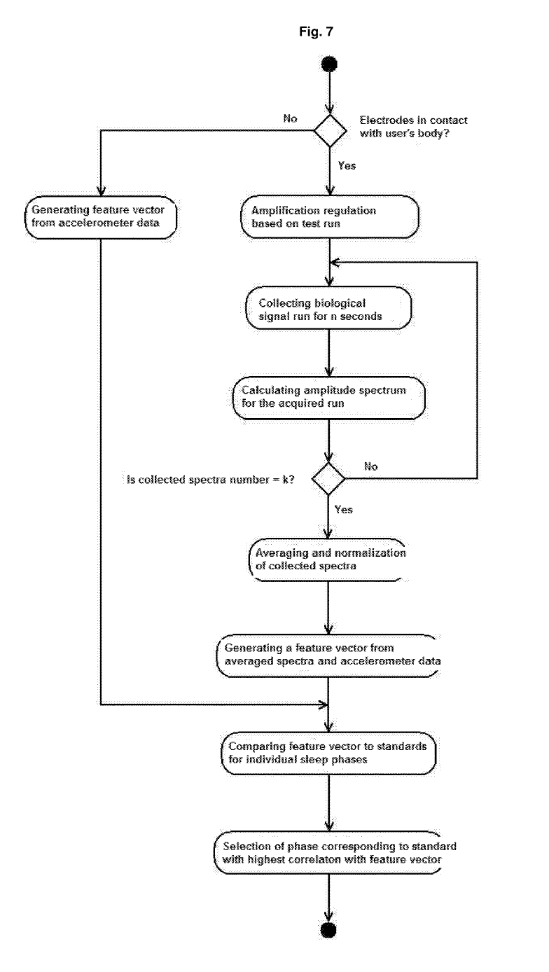

The method of operation of the system according to the invention and sleep phase classification is schematically illustrated on FIG. 6 and FIG. 7. After system activation the device initializes and passes from standby mode to the awaiting-connection mode. It remains in this mode until connection is established with an external device, e.g. via Bluetooth. After establishing connection the device waits for configuration data: the waking hour, duration of the waking buffer, the waking method. Following this stage, the system is ready to begin measurements. First of all, the device waits for correct mounting on the head by the user. For this purpose, the electrodes contact with the skin of the studied person is tested. During the electrodes contact test, a rectangular high-frequency signal is supplied to the ground electrode. The test is considered positive if the signal received from the remaining electrodes is consistent with the test signal. In case of an unsuccessful test, it is repeated.

Following the completed test the device begins a measurement of a bioelectric signal and sleep phase classification. Firstly, the electrodes test is performed, to confirm whether the device has not moved on the studied person's head rendering signal measurement impossible. If this test is unsuccessful, sleep phase is determined based on accelerometer data only. Otherwise, amplification by the biological amplifier is regulated based on a short test run: based on a signal fragment, amplification for the system is selected so that the maximal signal value does not exceed the acceptable voltage value for the analog-to-digital converter. When amplification is set, an n-second signal fragment acquisition is possible (n is a parameter of the algorithm), on the basis of which an amplitude spectrum is calculated using the FFT algorithm.

This function is performed k-times (k is a parameter), whereupon the calculated spectra are averaged and normalized.

Regardless of the electrodes test outcome, a feature vector is generated, containing information on the user's movement frequency collected by the accelerometer. Furthermore, if the electrodes test is positive, information is added to the feature vector in form of a normalized and averaged amplitude spectrum. Based on the generated feature vector the current sleep phase for the studied person is determined. This is done by selecting the phase, whose standard has the highest correlation with the calculated feature vector. Standards for each phase have been determined based on the training data set and recorded in the classification algorithm in the device.

The described algorithm for sleep phase determination is run until waking is commenced. This is commenced after the first REM phase detected during the course of the waking buffer or after reaching operation limit for the device. After activation of waking, the data relating to determined sleep phases are sent to an external device.

The last step of the algorithm is bringing the device into sleep mode. The device may be awakened by moving and generating an event from the accelerometer. After awaking the algorithm switches back to the beginning of the operation algorithm.

Beneficial Effects of the Invention

Due to the use of the invention it is possible to avoid a very unpleasant, burdensome and unhealthy adaptive phase to polyphasic sleep, wherein there is a significant sleep deficit before the body switches to the new mode.

The invention will monitor polyphasic sleep efficacy, allowing to avoid significant sleep deficits and ineffectiveness of polyphasic sleep. If the polyphasic sleep mode is ineffective the system will automatically propose a different one—more suited to the users' body.

A beneficial effect is also waking the user in a suitable sleep phase (REM), resulting in much more pleasant and smooth passing from the state of sleep to the state of wakefulness.

Polyphasic sleep efficiency involves the REM sleep content during the whole sleep and subjective feelings of the patient after 2 weeks from commencing sleeping in polyphasic mode. If the naps of a person in polyphasic sleep contain less than 90% of REM sleep, they are ineffective. If after 2 weeks from commencing sleeping in polyphasic mode, the person feels sluggish, drowsy then this indicates that the polyphasic sleep mode is not suited to the person's body.

1. A system for polyphasic sleep management, characterized in that it comprises components such as
electrodes for measuring a biological signal;
a biological amplifier for amplifying the biological signal;
a microcontroller for controlling individual system components, such as a biological amplifier and an accelerometer, as well as for communication with an external device;
an accelerometer for collecting data relating to user's movement frequency;
an external device, such as a computer, tablet and a mobile phone.

2. The system according to claim 1, characterized in that it comprises at least 3 electrodes, two frontal ones for measuring a biological signal, connected to the input of the biological amplifier and a central one connected to the ground of the amplifier.

3. The system according to claim 1, characterized in that it additionally comprises two zygomatic electrodes and two temporal electrodes.

4. The system according to claim 1, characterized in that in the biological amplifier the signal is initially high-pass filtered by the input followers and amplified; afterwards it is band-stop filtered with an active filter of the ‘double-T’ type, followed by low-pass filtering with a quaternary filter of Butterworth characteristic, which simultaneously additionally amplifies the signal, and the last amplifying stage is realized using a bilateral switch.

5. The system according to claim 1, characterized in that the microcontroller classifies the current sleep phase based on the biological signal received by the analog-to-digital converter and the data from the accelerometer, and sends the obtained results to the external device.

6. The system according to claim 1, characterized in that the microcontroller communicates with the external device via Bluetooth Low Energy (BLE) interface.

7. The system according to claim 1, characterized in that the accelerometer additionally wakes the microcontroller from sleep mode.

8. A device for sleep analysis, characterized in that it comprises:
a mask, acting as a carrier component, for mounting on the user's head;
at least one PCB plate (12,
at least three electrodes, two frontal ones (1), for measuring a biological signal connected to the input of a biological amplifier (11) and a central one (2) connected to the ground of the amplifier (11), and
a power source (9), such as an accumulator battery;
wherein the PCB plate (12) contains components such as
a microcontroller (5) for controlling individual system components, such as a biological amplifier (11) and an accelerometer (7), as well as for communication with an external device;
a biological amplifier (11) for amplifying the biological signal;
an accelerometer (7) for collecting data relating to user's movement frequency.

9. The device according to claim 8, characterized in that it additionally comprises an integrated antenna (6).

10. The device according to claim 8, characterized in that it additionally comprises at least one light source (8), such as a one-color LED, a multicolored RGB LED, a lightbulb.

11. The device according to claim 8, characterized in that it additionally comprises a vibrating component (10).

12. The device according to claim 8, characterized in that it additionally comprises two zygomatic electrodes (4) and two temporal electrodes (3).

13. The system according to claim 1, characterized in that the components such as:
the electrodes for measuring a biological signal;
the biological amplifier for amplifying the biological signal;
the microcontroller for controlling the individual system components such as the biological amplifier and the accelerometer, as well as for communication with an external device;
the accelerometer for collecting data relating to user's movement frequency;
are integrated in one device for sleep analysis, as defined in claim 8.

14. A method of operation of the system defined in claim 13, characterized in that it comprises steps, wherein:
the system is activated,
the device for sleep analysis is initialized;
connection is established between the device for sleep analysis and an external device, which subsequently sends configuration data such as the waking hour, duration of the waking buffer, the waking method;
the device for sleep analysis is mounted on the user's head;
electrodes contact with the user's skin is tested;
a biological signal is measured;
the current sleep phase is classified;
waking is commenced in the suitable sleep phase;
data is sent to the external device;
the device for sleep analysis is brought into sleep mode, wherein it awaits re-initialization.

15. The method according to claim 14, characterized in that the device for sleep analysis is initialized by an event from the accelerometer.

16. The method according to claim 14, characterized in that the connection between the device for sleep analysis and the external device is established using a wireless Bluetooth technology.

17. The method according to claim 14, characterized in that the waking is commenced after the first REM phase detected during the course of the waking buffer or after reaching operation limit for the device.

18. A method of current sleep phase classification, characterized in that it comprises steps, wherein:
electrodes test is performed in order to confirm the possibility of measuring a biological signal;
amplification by the biological amplifier is regulated based on a test run, so that the maximal signal value does not exceed the acceptable voltage value for the analog-to-digital converter;
an n-seconds signal fragment is acquired;
an amplitude spectrum for the acquired signal is calculated k-times using Fast Fourier Transform, FFT;
the calculated spectra are averaged and normalized;
a feature vector for the averaged and normalized spectra and the accelerometer data is generated;
the obtained feature vector is compared with standards for individual sleep phases;
the sleep phase corresponding to the standard with the highest correlation with the feature vector is selected.

19. The method according to claim 18, characterized in that in case of negative result of the electrodes test, a feature vector is generated only from accelerometer data, followed by comparison of the obtained feature vector with the standards for individual sleep phases and the sleep phase corresponding to the standard with the highest correlation with the feature vector is selected.

20. Use of the system according to claim 1 for polyphasic sleep management.

21. Use of the device according to claim 8 for polyphasic sleep management.
